(19) United States
(12) Patent Application Publication
(10) Pub. No.: US 2012/0204884 A1
(43) Pub. Date: Aug. 16, 2012

(54) Endotracheal tube cuff pressure regulator

(75) Inventor: William R. Howard, North Easton, MA (US)

(73) Assignee: Outcome Solutions LLC

(21) Appl. No.: 12/931,775

(22) Filed: Feb. 10, 2011

(51) Int. Cl. A61M 16/04

(52) U.S. Cl. 128/207.15

(57) ABSTRACT

An inflated endotracheal tube cuff maintained at a desired inflation using a constant air source. The constant air flow source is connected to a pressure regulator that is connected to the endotracheal tube cuff via the endotracheal tube cuff lumen. The regulator includes a bleed valve used to control the air pressure to the cuff so that a constant desired air pressure is maintained. The regulator may also include a manometer for visually displaying the pressure in the regulator, and thereby the cuff.

### BACKGROUND OF THE INVENTION

(1) Field of the Invention

This invention relates to endotracheal tube systems, and more particularly to a method and apparatus for maintaining inflation of the cuff associated with the endotracheal tube at a desired inflation pressure utilizing a constant air flow from a pressurized air supply, such as the air discharge outlet commonly found in hospital rooms.

(2) Description of the Prior Art

In the treatment of patients requiring breathing assistance, the use of an endotracheal tube for insertion into the trachea of the patient by way of the mouth, nose or a surgically created opening is common. The endotracheal tube is connected to a breathing apparatus such as a respirator which operates on repeated cycles of alternative inspiration and expiration. Upon inspiration, air or anesthesia or oxygen is supplied under pressure to the lungs, and upon expiration the air is allowed to exhaust from the lungs. The tube is typically provided with an annular, inflatable cuff to provide a seal between the trachea and the tube when air under pressure is supplied by the respirator through the endotracheal tube into the lungs.

The endotracheal cuff is generally inflated after being placed into the trachea to provide a seal against the interior wall of the trachea. Typically, the cuff is inflated by means of a conventional syringe which forces air into the cuff through a relatively small diameter secondary tube, known as a lumen or tubule, provided in the wall of the endotracheal tube. Examples of such cuffs are shown in U.S. Pat. Nos. 3,402,718; 3,504,676; 3,565,079; 3,642,005; 3,693,624; 3,794,036; 3,794,043; 3,848,605; and 3,901,246.

Because the cuff is continuously inflated, inspiration and expiration is forced to occur through the endotracheal tube. An advantage of the cuffed endotracheal tube is to prevent the in-flow of vomitus from the esophagus into the trachea. The passage of fluids into the lungs also tends to allow for the passage of bacteria causing ventilator-associated pneumonia (VAP). A disadvantage of the continuous pressurization of the cuff is that the cuff causes sloughing of the bronchial cells, and the pressure might disturb normal blood flow thus causing, in extreme cases, the cells to become necrotic. On the other hand, if the pressure of the cuff is too low a pressure, the artificial respiration can be inhibited due to a leakage of anesthetic gas, oxygen or air. Other systems have been developed which reduce the inflation pressure of the cuff during expiration (U.S. Pat. Nos. 4,119 and 4,501,273).

Another disadvantage of the traditional cuff is that clinicians must intermittently check the cuff to avoid loss of cuff volume and integrity of the tracheal seal since, over time, the cuff loses its pressure. The cuff often has to be re-inflated to assure proper respiration. Thus, there is need for a convenient way to automatically maintain a cuff inflation at a desired inflation pressure.

### SUMMARY OF THE INVENTION

The present invention is directed to a system and method for maintaining endotracheal cuff inflation with a source of constant air flow, eliminating the need to periodically reinflate the cuff with a syringe as in previous methods. Generally, the system is comprised of a pressure regulator adapted for attachment to a source of constant air flow, such as the air supply outlet of a hospital room, and to a tube connecting the regulator to an endotracheal cuff.

In general, the pressure regulator of the invention is comprised of a regulator housing enclosing an air chamber, a means for releasing excess air from the chamber to maintain a constant pressure, e.g., an adjustable pressure valve, referred to herein as a bleed valve or PEEP valve, and preferably a means to visually display the air pressure, such as a manometer.

The regulator housing includes openings extending from the air chamber to the housing exterior to connect other components. Specifically, an air supply opening is provided for attachment of the regulator to a constant air flow source; a valve opening is provided for attachment of the bleed valve; a manometer opening is provided for attachment of the manometer; and an air tubing opening is provided for attachment of the cuff.

The housing may be shaped at each tube opening to accommodate attachment of the component and optimize performance of the regulator. For example, the air supply opening may be in the form of a tubular stem that extends upwardly from the housing, with the stem being open to the chamber.

The bleed valve opening, preferable in the lower part of the housing opposite the flowmeter opening, may be a generally cylindrical opening sized to receive a tubular component of the bleed valve to be described in detail hereinafter. The manometer opening may be an opening in the wall of the housing that extends through the rear wall of the manometer housing. Preferably, the regulator housing and manometer housing are integrally formed. The air tubing opening may also be a tubular stem in communication with the chamber and extending outwardly from the chamber housing wall, with the outer diameter of the stem being sized for insertion of the proximal end of an air tube over the tubular stem.

The bleed valve is comprised of a tubular member, preferably cylindrical, having an outer diameter sized for snug insertion into the bleed valve opening of the chamber housing, a valve plug slidable within the lower part of the tubular member, a spring urging the plug upwardly in the tubular member, and an adjustable end cap to adjust the spring compression.

The tubular member includes an inner flange intermediate its upper and lower ends, and one or more air escape openings in the tubular member wall below the inner flange. The plug has a diameter approximately equal to the inner diameter of the tubular member but greater than the inner diameter of the flange. As a result, the plug can only slide upwardly in the tubular member until it engages the flange.

The air escape opening, which may be in multiple sections, is located immediately beneath the flange. When the plug is pressed upwardly against the flange, air is prevented from escaping from the housing air chamber. The pressure exerted by the plug against the flange is controlled by the spring and the adjustable spring compression means. As the compression means is tightened, greater pressure is exerted on the plug, requiring greater pressure to separate the plug from the flange and allow air to escape.

As long as the pressure on the plug exceeds the air pressure within the chamber, no air escapes through the air escape openings. However, when the air pressure within the chamber is greater than the spring pressure on the plug, the plug is forced away from the flange, allowing air to escape, equalizing the spring and air pressure.

The amount of air pressure within the chamber can be visually ascertained by viewing the manometer. The manometer is generally comprised of a manometer housing with a transparent face, a circular dial with indicia indicative of air pressure, a pointer rotatable around the dial, and means for rotating the pointer responsive to changes in pressure within the air housing chamber.

In one embodiment of a manometer, the pointer may be mounted on a shaft having a spiral protrusion, with the shaft being mounted in an opening in a moveable wall or diaphragm that moves toward the manometer dial as the pressure increases within the regulator housing. The opening includes a slot to receive the spiral protrusion so that the shaft, and thereby the pointer, rotates upon movement of the wall or diaphragm. Movement of the diaphragm acts against a spring which returns the diaphragm toward its initial position as pressure decreases.

Air under regulated pressure is discharged through the air tubing opening and conveyed to the endotracheal tube cuff. Normally, a connector tube will be used to connect the regulator to an endotracheal tube cuff inflation tube or lumen.

The air flow rate from some air supplies, such as the air supply obtained from the air supply outlet of a hospital room, may be greater that is required to maintain cuff inflation. If so, the air supply may be discharged through a flowmeter to reduce the air flow to an acceptable level. If a flowmeter is present, the regulator of the present invention is attached to the flowmeter instead of directly to the air supply outlet, with a flowmeter connector being used to connect the flowmeter and regulator, The flowmeter connector may include a first sleeve for attachment to the regulator and a second sleeve to receive the discharge end of a flowmeter. For ease of connection to some flowmeter connectors, the second sleeve may be flared.

The flowmeter contemplated by the present invention is of the type commonly found in hospitals for use in controlling the flow of oxygen or air from outlets in patients' rooms. These flowmeters include a first connector to releasibly attach the flowmeter to the air flow source, a second connector to attach the flowmeter to medical tubing or another device, and a flow control means, such as a rotatable knob, to control the amount of oxygen or air flowing through the flowmeter. The flowmeter also commonly includes a gauge to visually indicate the flow rate. For example, the flowmeter can include a transparent housing with a float inside the housing that moves up or down dependent on the flow rate, and indicia on the side of the housing to show the float position.

In operation, the regulator is attached directly to the air source outlet, or to the discharge end of the flowmeter, which is connected to the source of constant air flow. The regulator is connected via the air tubing to the endotracheal tube cuff The regulator is then adjusted so that the pressure within the cuff is at the desired level.

### BRIEF DESCRIPTION OF THE DRAWINGS

FIG. 1 is a front view of the regulator, a flowmeter, and endotracheal tube.

FIG. 2 is a side view of the regulator.

FIG. 3 is a partial sectional side view of the regulator.

### DETAILED DESCRIPTION OF THE INVENTION

In the following description, terms such as horizontal, upright, vertical, above, below, beneath, and the like, are used solely for the purpose of clarity in illustrating the invention, and should not be taken as words of limitation. The drawings are for the purpose of illustrating the invention and are not intended to be to scale.

In the preferred embodiment described and illustrated, the regulator is connected through a flowmeter to the air source, since a flowmeter will be normally used to reduce the air flow to an acceptable level, e.g., for about 1 liters/minute to about 3 liters/minute. It will be understood, however, that the regulator may be connected directly to a constant air supply source which is at an acceptable air flow volume.

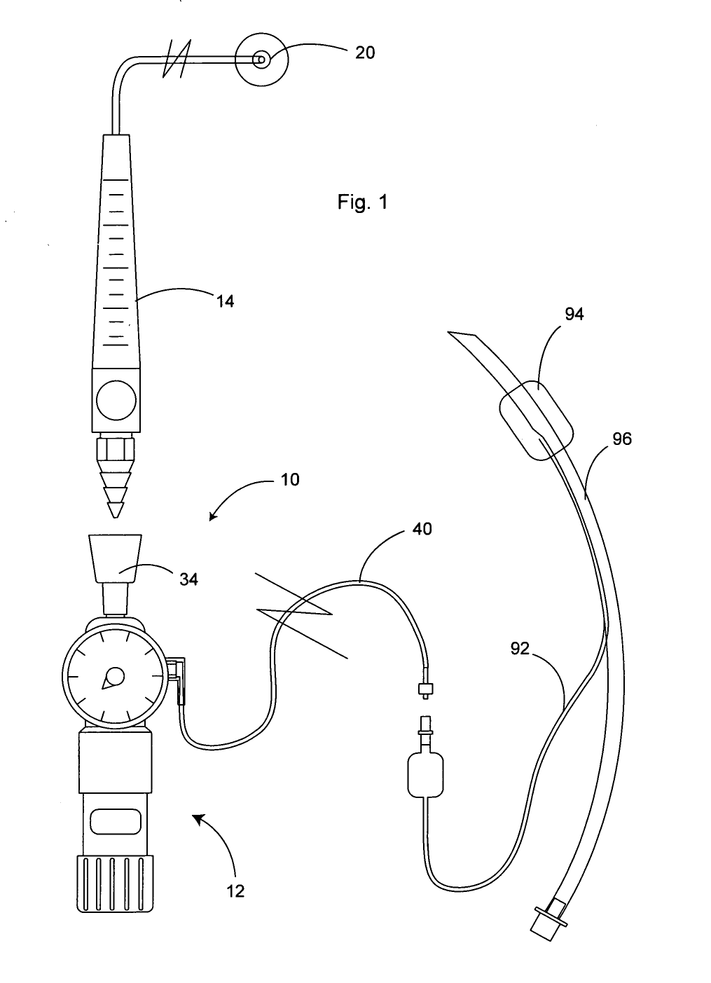

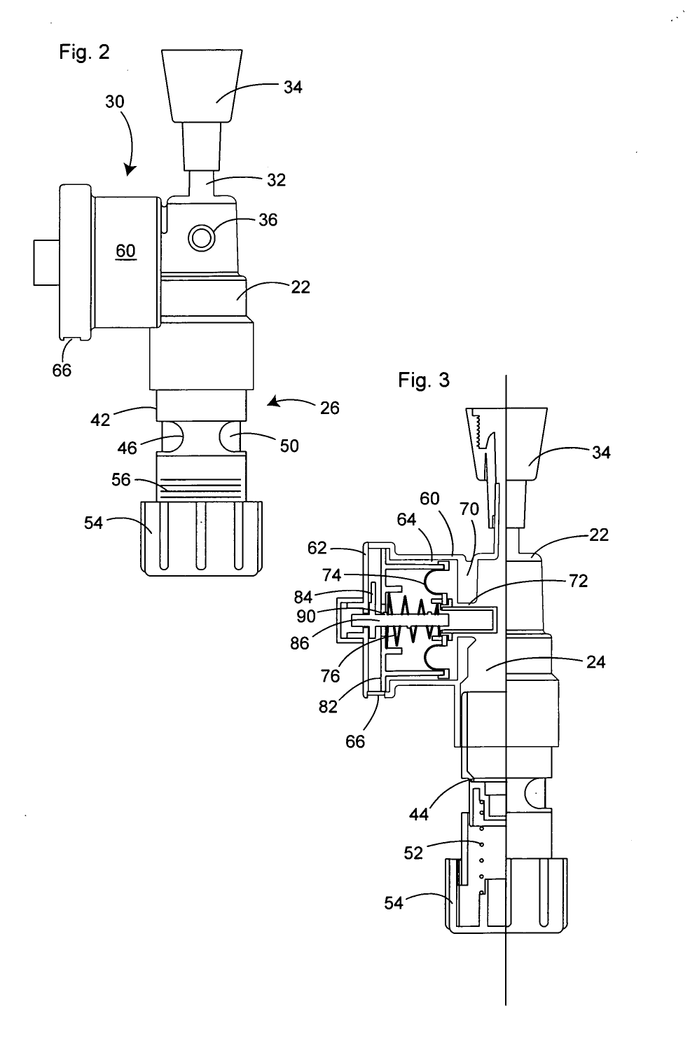

As best shown in FIG. 1, the preferred embodiment of the present system, generally 10, connects regulator, generally 12, between flowmeter, generally 14, and endotracheal tube, generally 16. Flowmeter 14 is in communication with a constant air source 20, such as a wall outlet of the type often found in hospital rooms.

Regulator 12 is comprised of housing 22 enclosing an air chamber 24, a bleed valve 26 to control the air pressure within chamber 24, and a manometer 30 to visually display the air pressure within chamber 24. Housing 22 includes a first tubular stem 32 to attach flowmeter connector 34 and a second tubular stem 36 to connect air tubing 40.

Bleed valve 26 is comprised of a cylindrical tubular conduit 42 having an intermediate inner flange 44 and an air escape opening 46 immediately beneath flange 44. Valve 26 also includes a valve plug 50 snugly slidable in conduit 42 beneath flange 44, a spring 52 urging plug 50 into engagement with flange 44, and a threaded cap 54 to adjust the compression of spring 52. Conduit 42 also includes indicia 56 to show the position of cap 54.

Manometer 30 is comprised of manometer housing 60 with transparent faceplate 62 enclosing manometer chamber 64, which is open to atmospheric pressure via gap 66. The rear wall 70 of manometer 30 is common with housing 22 and includes opening 72 to allow air to flow from air chamber 24 into chamber 64. Chamber 64 includes a moveable diaphragm plate 74 that is urged toward rear wall 70 by spring 76.

Manometer 30 also includes a dial 82 with indicia showing air pressure beneath faceplate 62. A rotatable pointer 84 is mounted on shaft 86 and is positioned to rotate around a pathway between dial 82 and faceplate 62. Shaft 86, which includes spiral protrusion 90, extends through shaft opening 80 with protrusion 90 fitting within the slot.

In operation, regulator 12 is attached to flowmeter 14 with connector 34, so that a constant air volume flows into regulator air chamber 24, and from chamber 24 through air tubing 40 and lumen 92 to inflate endotracheal tube cuff 94 alongside endotracheal tube 96. Bleed valve 26 is used to adjust air pressure within chamber 24 and thereby cuff 96. Air pressure within chamber 24 is read with manometer 30.

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. A regulator for use in maintaining a constant inflation of an endotracheal tube cuff using a constant air flow source comprising:
a) a regulator housing enclosing an air chamber, said housing including a first opening in communication with said air chamber adapted for attachment to said constant air flow source and a second opening in communication with said chamber for attachment to an endotracheal cuff; and
b) means for controlling the air pressure in said air chamber.

2. The regulator of claim 1, further comprising a means to display the air pressure within said air chamber.

3. The regulator of claim 1, wherein said means for controlling the air pressure in said air chamber is a bleed valve.

4. The regulator of claim 1, further including a flowmeter connector adapted to attach a flowmeter to said first opening.

5. The regulator of claim 1, wherein said constant air flow source includes a wall outlet.

6. The regulator of claim 1, further including flexible tubing having a proximal end attachable to said regulator housing and a distal end attachable to the lumen of an endotracheal tube.

7. The regulator of claim 3, wherein said bleed valve is comprised of a tubular conduit attachable to said regulator housing in communication with said air chamber, said conduit having an internal flange and an air escape opening immediately below said flange; a plug slidable within said conduit; a spring urging said plug against said flange; and means for adjusting the compression of said spring, and thereby the pressure of said plug against said flange, wherein air escapes from said air chamber through said air escape opening when the air pressure in said air chamber exceeds the pressure exerted by said spring.

8. The regulator of claim 2, wherein said means to display air pressure is a manometer comprised of a manometer housing with a transparent face, a dial, a rotatable pointer, and a diaphragm plate moveable with changes in the air pressure in said air chamber, said pointer rotating responsive to movement of said diaphragm plate.

9. A system for maintaining inflation in an endotracheal tube cuff comprising:
a) a constant air flow source having an outlet;
c) an air pressure regulator in communication with said air source, said regulator including an exhaust opening adapted to discharge air to an endotracheal tube cuff.

10. The system of claim 9, further including a flowmeter connecting said air source outlet and said regulator.

11. The system of claim 10, further including a connector to attach said flowmeter to said regulator.

12. The system of claim 8, wherein said regulator comprises a regulator housing enclosing an air chamber, and a bleed valve for controlling the air pressure in said air chamber.

13. The system of claim 12, wherein said regulator further includes a manometer for displaying the air pressure within said air chamber.

14. A method for maintaining a constant pressure in an inflated endotracheal tube cuff comprising:
a) providing a source of constant air flow;
b providing a pressure regulator;
c) connecting said regulator to said air flow source;
d) connecting said regulator to said cuff, whereby pressurized air flows from said air flow source to said cuff; and
e) adjusting the air pressure with said regulator so that air at a predetermined pressure is constantly maintained in said cuff.

15. The method of claim 14, further including providing a flowmeter, and connecting said flowmeter to said air source and said regulator to said flowmeter.

16. The method of claim 15, further including providing a flowmeter connector, and connecting said flowmeter and said regulator to said connector.

17. The method of claim 14, wherein said cuff includes a lumen and said method further includes providing flexible tubing and connecting said regulator to said lumen with said tubing.

18. The method of claim 14, wherein said regulator is comprised of a regulator housing enclosing an air chamber; an air source connector adapted to connect said regulator to said air source; a bleed valve for controlling the air pressure in said air chamber; and a manometer for displaying the air pressure within said air chamber.

19. The method of claim 14, wherein said bleed valve is comprised of a tubular conduit attachable to said regulator housing in communication with said air chamber, said conduit having an internal flange and an air escape opening immediately below said flange; a plug slidable within said conduit; a spring urging said plug against said flange; and means for adjusting the compression of said spring, and thereby the pressure of said plug against said flange, wherein air escapes from said air chamber through said air escape opening when the air pressure in said air chamber exceeds the pressure exerted by said spring.

20. The method of claim 14, wherein said air flow is at a volume of from about 1 liters/minute to about 3 liters/minute.
